(19) United States
(12) Patent Application Publication
(10) Pub. No.: US 2015/0057640 A1
(43) Pub. Date: Feb. 26, 2015

(54) Circular bandage for drain tube

(71) Applicant: Evelyn M. Tarkington, Boswell, IN (US)

(72) Inventor: Evelyn M. Tarkington, Boswell, IN (US)

(21) Appl. No.: 14/465,692

(22) Filed: Aug. 21, 2014

Related U.S. Application Data
(60) Provisional application No. 61/868,809, filed on Aug. 22, 2013.

(51) Int. Cl. A61F 13/02

(52) U.S. Cl. CPC A61F 13/0216; A61F 2013/00272
USPC 604/541

(57) ABSTRACT

A circular bandage features a small, circular aperture slightly offset from the center and a slit extending from the aperture, across the center of the bandage to the opposite edge. The bandage is provided in various sizes and thicknesses to accommodate wounds and incisions of various sizes, and features gauze of varying thicknesses to absorb excess drainage, and protect the user's clothing, bed sheets, and furniture upholstery. The bandage may also feature a flexible backing that extends outward around the edge of the gauze to provide an adhesive edge which adheres to the skin.

### CROSS-REFERENCE TO RELATED APPLICATIONS

This application claims the benefit of U.S. Provisional Application No. 61/868,809, filed Aug. 22, 2013, which is hereby incorporated by reference.

### STATEMENT REGARDING FEDERALLY SPONSORED RESEARCH OR DEVELOPMENT

Not Applicable

### PARTIES TO A JOINT RESEARCH AGREEMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates generally to medical supplies, and in particular to a circular bandage for a drainage tube. The insertion of drainage tubes, feeding tubes, tracheotomy tubes, and similar devices into wounds and surgical incisions has created a unique problem for medical caregivers. Excess drainage from the wound or incision must be absorbed, and the wound or incision must be kept clean to prevent infection. Various dressings have been applied to these sites, but have generally proven to be unsatisfactory. They must be changed frequently, but the inserted tube must not be disturbed. A circular adhesive or non-adhesive bandage, featuring a small, circular aperture slightly offset from the center and a slit extending from the aperture, across the center of the bandage to the opposite edge, would resolve this problem. The bandage may be used by medical offices, hospitals, nursing homes, home health care professionals, and veterinary clinics, and is provided in various sizes and thicknesses to accommodate wounds and incisions of various sizes. The bandage features thick gauze to absorb any excess drainage, and protect the user's clothing, bed sheets, and furniture upholstery. The bandage may also feature a flexible backing that extends outward around the edge of the gauze to provide an adhesive edge which safely adheres to the skin. This bandage may help the patient recovering from an injury or surgery to recover more rapidly, and lead a more normal lifestyle during recovery.

### SUMMARY OF THE INVENTION

Accordingly, the invention is directed to a circular bandage, featuring a small, circular aperture slightly offset from the center and a slit extending from the aperture, across the center of the bandage to the opposite edge. The bandage is provided in various sizes and thicknesses to accommodate wounds and incisions of various sizes, and features gauze of varying thicknesses to absorb excess drainage, and protect the user's clothing, bed sheets, and furniture upholstery. The bandage may also feature a flexible backing that extends outward around the edge of the gauze to provide an adhesive edge which adheres to the skin.

Additional features and advantages of the invention will be set forth in the description which follows, and will be apparent from the description, or may be learned by practice of the invention. The foregoing general description and the following detailed description are exemplary and explanatory and are intended to provide further explanation of the invention.

### BRIEF DESCRIPTION OF THE DRAWINGS

The accompanying drawings are included to provide a further understanding of the invention and are incorporated into and constitute a part of the specification. They illustrate one embodiment of the invention and, together with the description, serve to explain the principles of the invention.

FIG. 1 shows a top perspective view of the first exemplary embodiment, displaying the bandage 10, the top side 10A of the bandage 10, the aperture 11, the slit 12, the gauze 13, and the adhesive edge 14.

FIG. 2 shows a top view of the first exemplary embodiment, displaying the bandage 10, the top side 10A of the bandage 10, the aperture 11, the slit 12, the gauze 13, and the adhesive edge 14.

FIG. 3 shows a side view of the first exemplary embodiment, displaying the bandage 10, the top side 10A of the bandage 10, the bottom side 10B of the bandage 10, the slit 12, the gauze 13, and the adhesive edge 14.

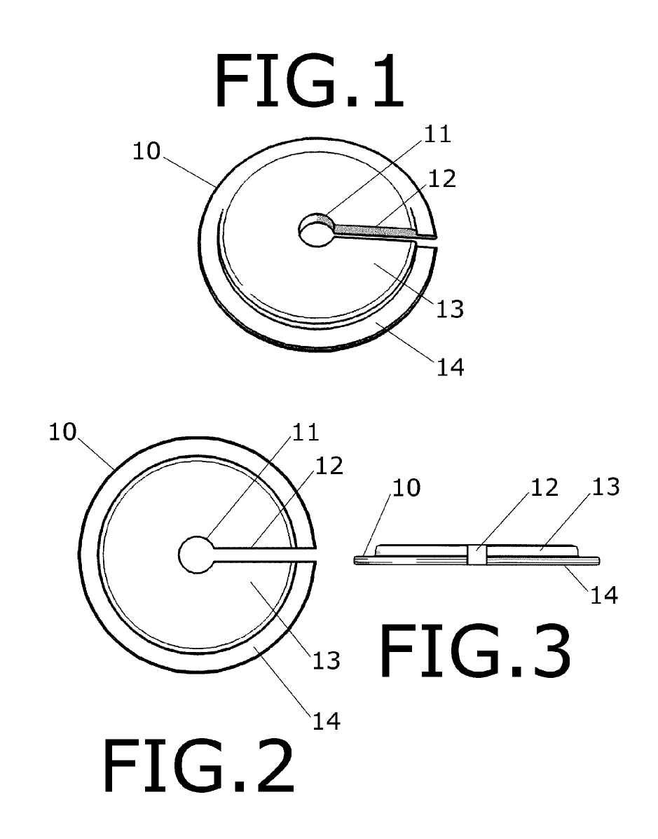

### DETAILED DESCRIPTION OF THE INVENTION

Referring now to the invention in more detail, the invention is directed to a circular bandage 10 having a top side 10A and a bottom side 10B, being pierced by a small, circular aperture 11 slightly offset from the center and being cut by a slit 12 extending from the aperture 11 radially across the center of the bandage 10 to the opposite edge. The bandage 10 features gauze 13 affixed to the bottom side 10B to absorb excess drainage, and protect the user's clothing, bed sheets, and furniture upholstery. The top side 10A of said bandage 10 is preferably formed of a flexible backing that extends outward around the perimeter of the gauze to provide an adhesive edge 14 which adheres to the skin.

The bandage 10 is provided in various sizes and thicknesses to accommodate wounds and incisions of various sizes. The bandage 10 is provided in sterile condition, and is packed in packaging such as paper or foil, individually in a box containing multiple bandages 10. The adhesive provided on the adhesive edge 14 is preferably safe for use on skin, and peels away easily when the bandage 10 is changed. In some cases, caregivers may choose to insert one or two additional gauze pads underneath the bandage 10 for additional absorption.

To use the first exemplary embodiment, the user must first remove any previous dressing from the wound or incision area and clean the area. If the user is using an additional gauze pad under the bandage 10 for additional absorption, the gauze pad must be placed on the wound or incision at this time. The user must then remove the bandage 10 from the packaging, and remove the paper covering from the peel-and-stick adhesive surface on the adhesive edge 14. The user must then grasp the tube that has been inserted into the wound or incision, and pass the tube through the slit 12 and into the aperture 11, such that the bandage 10 encircles the tube with the adhesive surface facing down. The user then slides the bandage 10 gently down the tube to be adhered in a flat and smooth manner around the wound or incision area.

The bandage 10 preferably manufactured from flexible, durable materials that may be safely sterilized, such as plastic and elasticized cotton. The gauze 13 is preferably manufactured from a flexible, durable fabric that may be safely sterilized and will not become adhered to a wound or incision, such as cotton. The gauze 13 may be provided in a variety of thicknesses. The adhesive edge 14 features a peel-and-stick adhesive which is safe for use on human skin.

Components, component sizes, and materials listed above are preferable, but artisans will recognize that alternate components and materials could be selected without altering the scope of the invention.

While the foregoing written description of the invention enables one of ordinary skill to make and use what is presently considered to be the best mode thereof, those of ordinary skill in the art will understand and appreciate the existence of variations, combinations, and equivalents of the specific embodiment, method, and examples herein. The invention should, therefore, not be limited by the above described embodiment, method, and examples, but by all embodiments and methods within the scope and spirit of the invention.

I claim:

1. A bandage system for a medical tube comprising:
(a) a bandage;
(b) a gauze;
(c) said bandage having a top side and a bottom side;
(d) said top side of said bandage being formed of a flexible backing;
(d) said gauze being affixed to said bottom side of said bandage;
(e) an adhesive edge;
(f) said adhesive edge being formed by said top side of said bandage extending beyond the perimeter of said gauze;
(g) said bandage being pierced by an aperture;
(h) said bandage being cut by a slit;
(i) said slit extending from said aperture to the outer edge of said bandage.

2. The bandage system of claim 1 wherein said adhesive edge is of a peel-and-stick adhesive.

3. The bandage system of claim 1 wherein said aperture is positioned slightly off the center of said bandage, and wherein said slit extends through the center of said bandage.

4. The bandage system of claim 1 wherein said bandage is circular in shape.

5. The bandage system of claim 2 wherein said aperture is positioned slightly off the center of said bandage, and wherein said slit extends through the center of said bandage.

6. The bandage system of claim 2 wherein said bandage is circular in shape.

7. The bandage system of claim 3 wherein said bandage is circular in shape.

8. The bandage system of claim 5 wherein said bandage is circular in shape.

9. A method of use of the bandage system of claim 1 comprising:
(a) removing any existing dressings from the point of insertion of a medical tube into a patient;
(b) passing said medical tube through said slit and into said aperture of said bandage such that said adhesive edge is directed toward the body of said patient;
(c) sliding said bandage down said medical tube; and
(d) adhering said bandage to the body of said patient via said adhesive edge such that said bandage surrounds said medical tube.

10. A method of use of the bandage system of claim 2 comprising:
(a) removing any existing dressings from the point of insertion of a medical tube into a patient;
(b) passing said medical tube through said slit and into said aperture of said bandage such that said adhesive edge is directed toward the body of said patient;
(c) sliding said bandage down said medical tube; and
(d) adhering said bandage to the body of said patient via said adhesive edge such that said bandage surrounds said medical tube.

11. A method of use of the bandage system of claim 5 comprising:
(a) removing any existing dressings from the point of insertion of a medical tube into a patient;
(b) passing said medical tube through said slit and into said aperture of said bandage such that said adhesive edge is directed toward the body of said patient;
(c) sliding said bandage down said medical tube; and
(d) adhering said bandage to the body of said patient via said adhesive edge such that said bandage surrounds said medical tube.

12. A method of use of the bandage system of claim 8 comprising:
(a) removing any existing dressings from the point of insertion of a medical tube into a patient;
(b) passing said medical tube through said slit and into said aperture of said bandage such that said adhesive edge is directed toward the body of said patient;
(c) sliding said bandage down said medical tube; and
(d) adhering said bandage to the body of said patient via said adhesive edge such that said bandage surrounds said medical tube.

13. The method of claim 10 further comprising removing the backing from said peel-and-stick adhesive.

14. The method of claim 11 further comprising removing the backing from said peel-and-stick adhesive.

15. The method of claim 12 further comprising removing the backing from said peel-and-stick adhesive.

16. The method of claim 9 wherein said medical tube is a surgical drain tube.

17. The method of claim 10 wherein said medical tube is a surgical drain tube.

18. The method of claim 11 wherein said medical tube is a surgical drain tube.

19. The method of claim 12 wherein said medical tube is a surgical drain tube.

20. The method of claim 15 wherein said medical tube is a surgical drain tube.
